(19) United States
(12) Patent Application Publication
(10) Pub. No.: US 2013/0245653 A1
(43) Pub. Date: Sep. 19, 2013

(54) ZERO ARTIFACT VASCULAR CLIP METHOD AND APPARATUS

(71) Applicant: Craig Michael Litherland, Concord, NH (US)

(72) Inventor: Craig Michael Litherland, Concord, NH (US)

(21) Appl. No.: 13/846,727

(22) Filed: Mar. 18, 2013

Related U.S. Application Data
(60) Provisional application No. 61/612,766, filed on Mar. 19, 2012.

(51) Int. Cl. A61B 17/122

(52) U.S. Cl. CPC A61B 17/122
USPC 606/158

(57) ABSTRACT

The present invention relates to vascular clip made of biocompatible, non-metallic material that minimizes artifacts and obscuration of a diagnostic image developed using modalities such as CATSCAN, and MRI. The vascular clip is dimensionally comparable to metal clips, while maintaining sufficient clamping force to stop the flow of blood from an aneurysm, a subarachnoid hemorrhage or bleeding on the brain.

### CROSS REFERENCE TO RELATED APPLICATION

This application claims the benefit of U.S. Provisional Application No. 61/612,766 filed on Mar. 19, 2012 and entitled ZERO ARTIFACT ANEURYSM CLIP which is herein incorporated by reference.

### FIELD OF THE INVENTION

A vascular clip made of biocompatible, non-metallic material that minimizes artifacts and obscuration of a diagnostic image developed using modalities such as CATSCAN, and MRI. The vascular clip is dimensionally comparable to metal clips, while maintaining sufficient clamping force to stop the flow of blood from an aneurysm, a subarachnoid hemorrhage or bleeding on the brain.

### BACKGROUND

Vascular surgical clips like hemostatic clips and aneurysm clips are often used in surgery to ligate vessels to stop the flow of blood. Surgical clips are also used to interrupt or occlude the oviduct or vas deferens in sterilization procedures. The clips are often left in place permanently and within a period of time the ligated end of the vessel will close, that is, hemostasis or occlusion will occur.

Subarachnoid hemorrhage (SAH), or bleeding on the brain, is a significant and commonly encountered problem. Most cases of SAH are caused by leaking from arteries of the brain. There is a very high mortality and complication rate, with the vast majority of patients experiencing a medical complication which is potentially severe in approximately 40% of cases. Examples of such complications include strokes and re-bleeding, which leads to significant costs in ICU care and medical management. Approximately 1-5% of the United States population harbors brain aneurysms, and approximately 30,000 of these aneurysms rupture every year.

Current treatments include endovascular coiling, which uses the femoral artery in the leg to thread up to a brain aneurysm to deploy coils to clot the aneurysm, or clip ligation, which involves an open brain surgery to manually place a clip of metallic material across the neck of the aneurysm. Metal clips are most commonly made from metals or alloys of titanium, elgiloy or stainless steel. The clip is typically formed from metallic wires that are formed into a torsional spring. The resulting clip has a normally closed position that is under spring preload. Using surgical tools, such as clip appliers that hold the torsional spring open to place the clip about the vessel, the jaws clamp the vessel and the nature of the spring loaded metal stays clamped resisting any force by the vessel to expand or open up.

Major academic centers treat brain aneurysms with approximately a 50-50% split between clipping and coiling. Currently available clips are made of metals, which cause image artifacts in diagnostic modalities such as Computer Tomography (CT) and Magnetic Resonance Imaging (MRI). This issue can impede diagnosis and treatment of complications experienced by these patients, and may prevent accurate monitoring of the aneurysm.

In particular, current MRI techniques exacerbate the interference properties of clips. For example, fast imaging techniques for MRI give rise to at least one order of magnitude in increased sensitivity to magnetic field inhomogenieties brought about by metallic clips. Field uniformities of one in 105 are preferred, but metal clips, particularly stainless steel clips, can reduce the homogeneity in the locality of the clip by orders of magnitude. Interferences are also seen using CT imaging techniques. Virtually all treated patients will require a post-operative MRI or CT scan to evaluate the aneurysm or a medical complication. Currently surgeons are severely limited in their ability to provide adequate care for this very dangerous problem.

A large majority of patients with brain aneurysms are amenable to surgical clipping, with the exclusion of patients who have aneurysms very deep in the posterior blood circulation of the brain, or the base of the skull, both of which are difficult to reach with a surgical approach. In addition aneurysms where the ratio of the neck diameter to that of the largest dome of the aneurysm is greater than about 0.5 inches are more amenable to vascular coiling.

There has been a continuous effort to minimize MRI artifact throughout the evolution of clip technology. Initially clips were made with steels that had some magnetic properties; these were dangerous because they could be forced to move by the magnetic field created by the MRI machine. Next, clips were made with non-magnetic steels that would not physically interact with MRI, but still obscure images. The latest designs use titanium which has less MRI imaging artifact than steels; but these clips still obscure images especially where the surgeon is trying to examine small features in the vasculature. For example, in the last decade there was an attempt to make a ceramic clip which would be MRI-invisible (see US Patent Application No. 2008/0004637 A1). However, a spring element made of titanium had to be incorporated in order to hold the ceramic jaws together because ceramic is not a viable material for springs as it does not carry tensile forces.

It is therefore, desirable to produce a small, biocompatible, polymeric vascular clip.

### OBJECTS AND SUMMARY OF THE INVENTION

The vascular surgical clip of the present invention is made of biocompatible material and accordingly minimizes interference with image diagnostic modalities such as CATSCAN and MRI. At the same time, the vascular clip is nearly the same size as comparable metal clips, while maintaining sufficient strength and possessing high reliability in the clip's latching mechanism in the closed position. The clip is configured to provide a secure means of handling and application to avoid premature release from an applier.

In a first embodiment, the vascular clip of the present invention uses PEEK, or polyether ether ketone, a substance currently used extensively in orthopedic and spine surgeries. As noted above, monitoring clipped aneurysms, and diagnosing and treating post-operative complications, is often inhibited because of the extensive artifact caused on MRI and CT images. An artifact or interference caused by a metallic clip in a diagnostic or post-operative MRI or CT image is an obscuration that makes it difficult to see the anatomical features of the image and therefore diagnose proper cessation of bleeding of a vessel, and/or other post-operative complications. The minimal interference or zero artifact clip properties of the present invention could greatly improve the way SAH and aneurysm patients are treated and significantly reduce the overall cost both of treating an aneurysm, and these post-operative complications.

The present invention of a vascular clip is essentially invisible under imaging (MRI) because it is made of biocompatible plastic. All vascular clips used for aneurysms on the market are made of metal because the designs employ a preloaded torsional spring that provides the necessary clamping force to cease blood flow and permanently affix the clip to the vessel. In order to generate this required clamping force using a spring-based design, a material with the stiffness and spring characteristics of a metal (e.g. titanium or steel) is required. The design of the present invention departs from the conventional torsional spring configuration and employs a snap-together configuration wherein the clip has a flexible frame member and rigid clamping member. The flexible frame, when squeezed by the surgeon's tool, provides for securing a tension member to a clasp to secure and clamp together the rigid clamping member with the required force. The tension member may further provide a clip applier access point to provide for the attachment of a surgical forceps or other tool to re-open and position the clip and then re-close and secure the vascular clip in the proper anatomical location to seal the vessel and stop blood flow. Alternative clasp mechanisms may use tensile fiber, such as dyneema with round or bulged ends to encircle the clip frame and clamp and secure the clip in a locked position.

In a first embodiment the vascular or aneurysm clip may be made from a biocompatible material such as PEEK (Polyether ether ketone), that is known and commonly used in long-term medical implants because of its mechanical strength and biocompatibility. Applications of PEEK include implants in orthopedics, spine, cardiovascular, and neurology—including deep brain stimulation. An inherent advantage in the use of biocompatible plastics in this design is a reduction in costs as compared to clips made of titanium and other metals. Conventional metal design clips are made in a precision fashion requiring hand craftsmanship with tight tolerances at minute dimensions. In comparison, injection molding of biocompatible plastics is inherently cheaper and scalable so that costs of goods may be a fraction of the metal clips currently available.

It is an object of the present invention to minimize or obviate interference and artifacts from MRI and CT imaging commonly seen in using metallic clips to stop blood flow.

It is another object of the present invention that a vascular clip is formed having a flexible member securing a clamping member with adequate force to clamp a vessel and permanently cease blood flow.

It is a further object of the present invention that the vascular clip be of a biocompatible material and comparably dimensioned to the metallic clips of the prior art.

It is a further object of the present invention that the vascular clip is manufactured from a plastic biocompatible material such as PEEK.

It is a further object of the present invention that the vascular clip provides a clamping force of between 50 to 500 grams of force and more specifically between 100 and 300 grams of force.

It is a further object of the invention that the latching mechanisms of the present vascular clip facilitates re-opening and re-closing of the mechanism to properly place and seal a vessel and to secure the clip in the proper position to permanently cease blood flow.

It is a still further object of the present invention that the vascular clip be manipulated by and releasable to re-position using a surgical clip applier.

The present invention is related to a re-attachable vascular clip comprising two jaws having clamping surfaces, two flexible members manipulating the two jaws to close, a tension member latching the two flexible members to lock the two jaws in a closed position; and wherein unlatching the tension member unlocks the two jaws. In the re-attachable vascular clip the tension member may be pivotably attached to at least one of the two flexible members. The tension member may comprise a first and second tension arm or a single tension arm. The re-attachable vascular clip may further have the tension member comprising opposing clasps. In the re-attachable vascular clip, the tension member may further comprise a clip actuator manipulating the tension member to latch and unlatch the two flexible members. In the re-attachable vascular clip the two jaws may close at a clamping force in a range of 50 to 500 grams of force and the vascular clip is a biocompatible plastic material which produces no imaging interference.

The present invention is further related to an aneurysm clip producing minimal interference in imaging comprising a compressible frame of a plastic material, a clamping member extending from the compressible frame; and wherein compressing the frame produces forces at the clamping member in a range of 50 to 500 grams of force. The aneurysm clip producing minimal interference in imaging may further comprise a latching member holding the frame in a compressed state and the latching member may release the frame from a compressed state. The latching member may further pivot from the frame. The latching member may further comprise first and second clasps. The aneurysm clip producing minimal interference in imaging may further comprise a frame of a substantially rectangular shape. The aneurysm clip producing minimal interference in imaging may further comprise a frame of a substantially elliptical shape. The clamping member may have one of at least a curved, rounded, and angled shape and may further comprise an angular extension.

The present invention is further related to a method of applying a zero artifact vascular clip to a vessel to cease blood flow, comprising the steps of locating the open jaws of a vascular clip around a vessel, closing the jaws of the vascular clip around the vessel, compressing a frame affixed to the jaws to apply adequate force to the vessel to cease blood flow, locking a tension member using an actuator of a clip applier to hold the frame in compression. The method of applying a zero artifact vascular clip to a vessel to cease blood flow may further comprise the steps of unlocking the tension member, decompressing the frame affixed to the jaws, opening the jaws of the vascular clip, repositioning the vascular clip around the vessel and closing the jaws of the vascular clip, compressing the frame affixed to the jaws to apply adequate force to the vessel to cease blood flow, locking the tension member using the actuator of the clip applier to hold the frame in compression.

These and other features, advantages and improvements according to this invention will be better understood by reference to the following detailed description and accompanying drawings.

### BRIEF DESCRIPTION OF THE DRAWINGS

Several embodiments of the present invention will now be described by way of example only, with reference to the accompanying drawings in which:

FIG. 1 is an embodiment of a vascular or aneurysm clip of the present invention in a closed and locked position clamping a vessel;

FIG. 2 is an exploded view of a vascular clip according to an embodiment of the present invention;

FIGS. 3A and 3B are detailed views of embodiments of hinges of an embodiment of the vascular clip of the present invention;

FIG. 4 is a perspective of an embodiment of the vascular clip of the present invention in an open state;

FIG. 5 is a side view of an embodiment of the vascular clip of the present invention with the jaws open;

FIG. 6 is a side view of an embodiment of the vascular clip of the present invention in the closed and latched position clamping a vessel and illustrating the forces acting on the frame and the vessel;

FIG. 7 is a side view of an embodiment of a vascular clip of the present invention in a closed but not latched position as held by a surgical tool;

FIG. 8 is a perspective view of an embodiment of the vascular clip of the present invention held by a surgical tool;

FIG. 9A is an embodiment of a vascular clip of the present invention in a closed but not latched position as held by a surgical tool;

FIG. 9B is an embodiment of a vascular clip of the present invention in a closed but not latched position as held by a surgical tool;

FIG. 10 is a further embodiment of a vascular clip of the present invention with a single tension arm in a prepared open position;

FIG. 11 is a side view of an embodiment of an upper deflection member of showing a pivot fastener extending from the interior surface of a deflection member of a vascular clip of the present invention;

FIG. 12 is a perspective view of an embodiment of the tension member as opposing clasps in a still further embodiment of a vascular clip of the invention in the open configuration;

FIG. 13 is a perspective view of an embodiment of the frame in an elliptical shape in a still further embodiment of a vascular clip of the present invention in the open state;

FIGS. 14A-14C illustrate the still further embodiment of a vascular clip of the present invention with the frame in an elliptical shape showing the clip in the open, partially closed, and closed and latched positions;

FIG. 15 is a perspective view of an embodiment of the vascular clip of the present invention with the frame in an elliptical shape in an open position;

FIG. 16 is a perspective view of an embodiment of the vascular clip of the present invention with the frame in an elliptical shape in a closed and unlocked position;

FIG. 17 is a perspective view of an embodiment of the vascular clip of the present invention with the frame in an elliptical shape in closed and locked position; and

FIGS. 18A-18G are top views of embodiments of optional jaw geometries of a vascular clip of the present invention.

### DETAILED DESCRIPTION OF THE INVENTION

The vascular clip of the present invention may be used in a number of applications to cease blood flow from a vessel in the human body. The zero artifact features make the vascular clip particularly well suited for the treatment of aneurysms within the brain. Aneurysms, such as subarachnoid type, in the brain are treated typically by coiling or clipping. The number of cases is divided approximately 50/50 between the two methods. Clipped aneurysms are of interest for this innovation. Clipping using a vascular or aneurysm clip blocks blood flow so that the aneurysm will clot and cease expanding so as not to burst or leak. The clip clamps the proximal blood vessel that feeds the aneurysm. Metallic clips of the prior art are made by various manufacturers with variations in size, shape, and holding/clamping force, but typically the designs are the same with a torsional spring and clamp or jaw. Forces of the jaw or clamp are on the order of approximately 100 to 300 grams of force. This is the force that is applied to the vessel to cease blood flow. Since the force of, for example, 100 grams of force is at a closed state with the jaws aligned and compressed together, the torsional spring of the prior art is preloaded during manufacture, that is, when provided to the surgeon they are in the normally closed position. The clips of the prior art are commonly made of titanium or steel or other alloys in order to accommodate the required preloading of the torsional spring. The metallic properties of these clips of the prior art can create the image artifacts and interferences that obscure accurate imaging of the brain vasculature, which requires detailed resolution. The non-metal MRI clip of the present invention has substantially zero effect on MRI and CT imaging.

An inherent design challenge using biocompatible polymers is developing enough clamping force w/o yielding the material such that it deforms out of place or loses stiffness. For this reason a plastic clip in a similar sized spring design as a metallic clip cannot produce sufficient clamping force because plastics are so low in modulus (stiffness) compared to metallic substances. The clamping force of a plastic clip of similar dimensions would be insufficient where acceptable forces may only be achieved by increasing the overall size which will be far too large to use as a vascular clip. For example carbon fiber reinforced peek (CFRP PEEK) has a Modulus of Elasticity o 18 GPa which is at the upper end of the range for polymers. In comparison, Titanium Ti G-4 commonly used in metallic clips has a modulus of 120 GPa which is about seven times that of CFRP PEEK.

In evaluating how the material stiffness will affect spring performance, the basic linear equation for the stiffness of a torsional spring demonstrates that the required forces could not be achieved within the dimensional requirements of approximately 3 mm-8 mm in diameter for the torsional spring in a surgical vascular clip.

Torsional Spring Stiffness
k=d⁴E/10.8DN
d=wire diameter
   E=Young's Modulus
   D=coil diameter
   N=# of coils

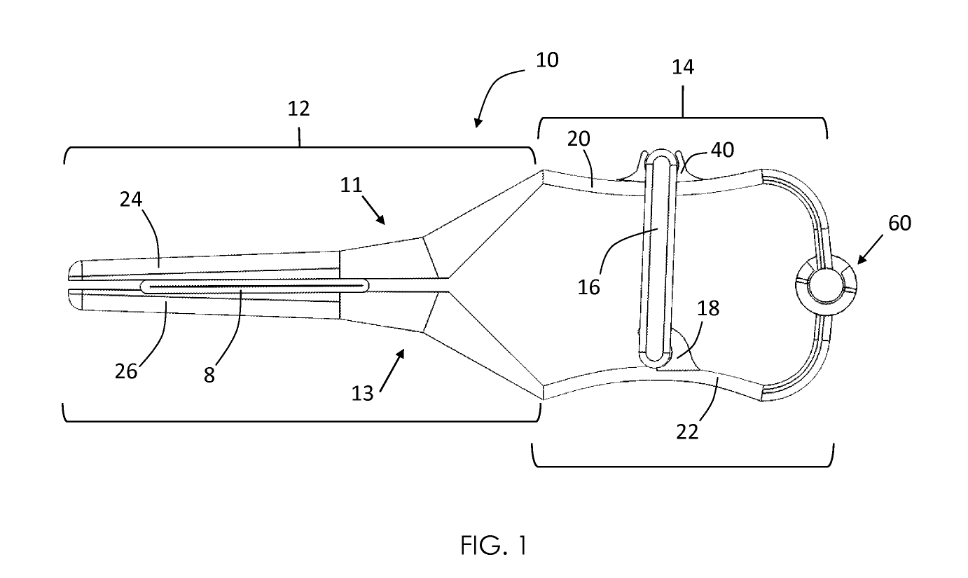

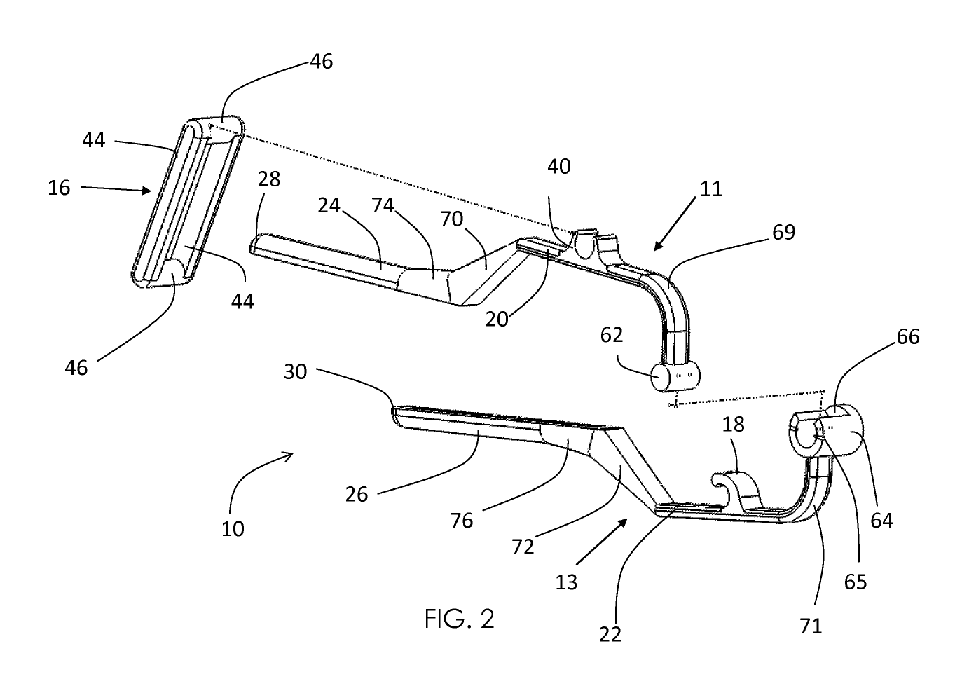

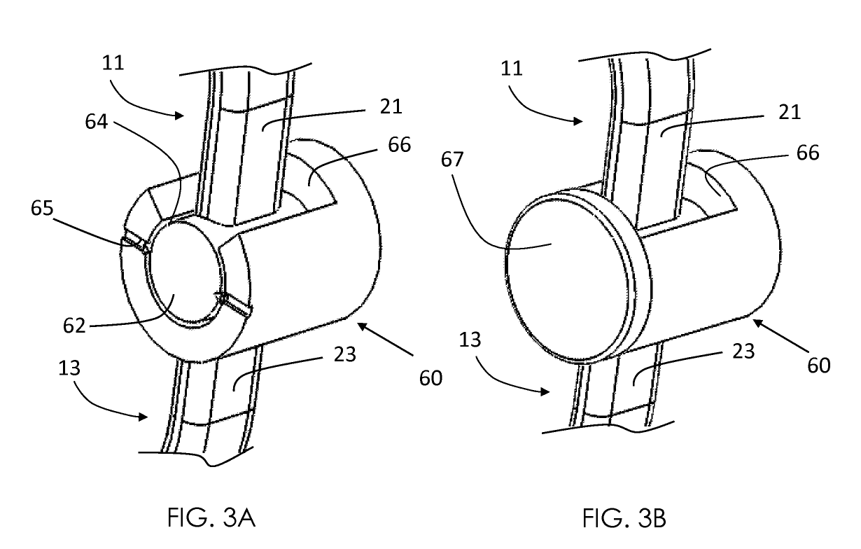

For a spring with the same geometry but made of CFRP PEEK there is therefore a loss of stiffness by a factor of at least 7. This analysis is based on the assumption that a spring could be molded in the same manner as a wound metal spring, which is difficult or impossible with current manufacturing technologies. Since a plastic torsional, or coil spring, analogous to the prior art is not feasible, flexural designs are considered. In the design of a first embodiment of the present invention and in contrast to the prior art, the clamping force as shown in FIG. 1 is caused by a flexible frame 14 formed using deflection beams 20 and 22 that are pulled towards one another using a tension member 16 and secured using a clasp 18 within the frame structure 14 to induce compression between the clamping members 12, thus holding the jaws 24 and 26 together. A vessel 8 is secured between the jaws 24 and 26 of the vascular clip 10 with sufficient force to seal the vessel and cease blood flow. The approximate force is in a range of 50 to 500 grams of force and more specifically between 100 and 300 grams of force. As shown in FIG. 2, the vascular clip 10 is formed with an upper clip member 11 that is affixed to a lower clip member 13 using a shaft 62 and barrel hinge 60 that provides for the clip members 11 and 13 to rotate within the hinge 60 and open and close the clamping member 12. The terms upper and lower, vertical and horizontal demonstrate a relationship of the elements and are not used to restrict the orientation or use of the present invention. The tubular shaft 62 of the upper clip member 11 slides in and is secured within the barrel receptor 64 of the hinge 60 on the lower clip member 13. A pivot abutment 66 provides for lateral adjustment of the shaft 62 within the receptor 64 in order to align the jaw members 24 and 26 and prevent misalignment or scissoring meaning that one jaw extends across the other jaw at an angle. By adjusting the jaw members 24 and 26 into alignment a greater surface area is available to contact and secure a vessel 8. After alignment, a retaining channel 65 as shown in FIG. 3A may be crimped, heated staked or otherwise treated to secure the clip member 11 in position within the barrel receptor 64. For example, by flowing material from the retaining channel 65 of the barrel receptor 64 over the shaft 62, the shaft 62 is retained but still allowed to rotate to open and close the clamping member 12. In a further embodiment as shown in FIG. 3B a cap 67 may be adhered to the barrel receptor 64 to retain the shaft 62 and align the jaw members 24 and 26 in parallel. It is within the scope of the present embodiment, and it will be understood by those skilled in the art, that there are various ways to retain a shaft within a receptor so as to form a rotatable hinge. For example in further embodiments, the shaft may be retained within the hinge using a shim or stopper to position and align the jaws 24 and 26 of the clamping member 12. The rotatable hinge 60 is positioned along a support structure of the frame 14 with an upper vertical support member 21 supporting the shaft 62 on clip member 11 and a lower vertical support member 23 supporting the barrel receptor 64 on clip member 13.

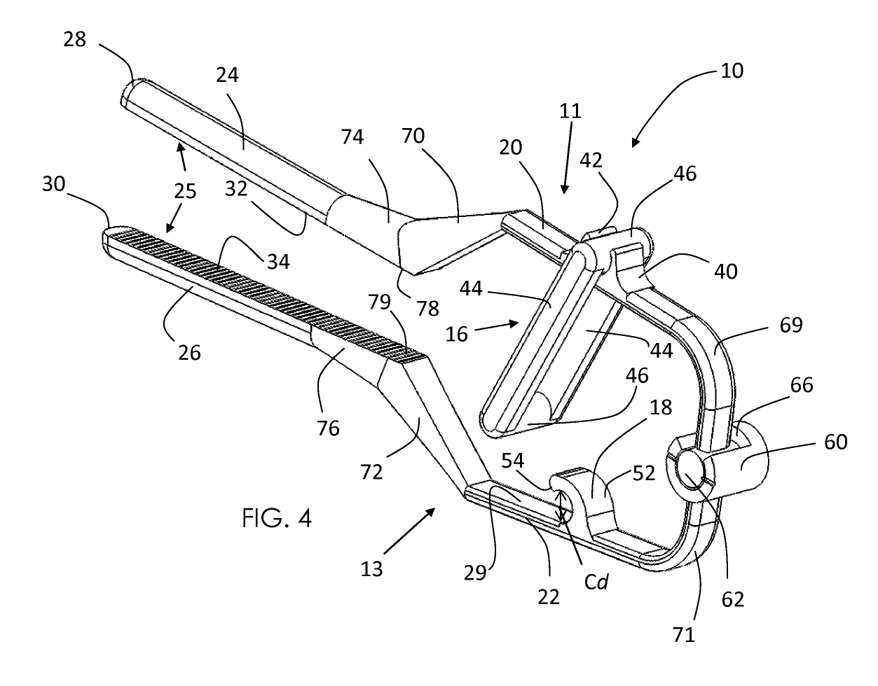

In assembly of the vascular clip 10, the tension member 16 may be installed by inserting the upper clamping member 11 through the tension arms 44 of the tension member 16 and affixing the tension member 16 to a pivot hinge 40 as described in further detail herein. In attaching the upper and lower members 11 and 13, the flexible frame 14 and clamp 12 are formed. The flexible frame 14 includes upper and lower deflection beams 20 and 22 connected through the vertical member supports 21 and 23 that includes, in this first embodiment, the rotatable hinge 60 consisting of the shaft 62 and barrel receptor 64. In further embodiments the upper and lower vertical supports 21 and 23 may be of a shortened length that would provide for a shortened length of the tension member 16 and an overall shortened vertical profile of the frame member 14 and in the range of 4 mm-8 mm in height. The upper and lower vertical supports 21 and 23 extend through curved supports 69 and 71 to attach each of the upper and lower deflection beams 20 and 22 as shown in FIG. 4. The curved structure is formed substantially perpendicular to the vertical support structure at an angle approaching 90° and in further embodiments it may be of any angle in a range of approximately 45° to 130°. The deflection beams 20 and 22 extend to upper and lower transition support members 70 and 72 forming the flexible frame 14. The deflection beams 20 and 22 may as well be of a longer dimensional length than shown providing for a lengthened horizontal profile of the frame member 14 and in the range of 7 mm-15 mm in length.

Extending from the rigid transition support members 70 and 72, upper and lower stiffening members 74 and 76 extend to the upper and lower jaws 24 and 26. The transition and support members 70 and 72, the stiffening members 74 and 76, and the jaws 24 and 26 form the clamp 12 of the vascular clip 10. Different from the two deflection beams 20 and 22, the transition support members 70 and 72 and the upper and lower stiffening members 74 and 76 may be of a thicker dimension to rigidly extend and support the upper and lower jaws 24 and 26 without substantially flexing or deforming when the deflection members 20 and 22 are in compression as described herein.

As shown in FIG. 2, in the exploded view, the tip 28 of the upper jaw leg 24 is inserted through the legs 44 of the tension member 16 and the tension member 16 is slid along the jaw leg 24 and jaw support member 74 and transition member 70 to the pivot hinge 40. The barrel cylinder 46 or other attachment mechanism of the tension member 16 is snapped or otherwise affixed to the pivot hinge 40. The cylindrical shape of the barrel 46 and extending prongs 42 of the hinge 40 allow the tension member 16 to rotate and swing freely within the frame handle 14.

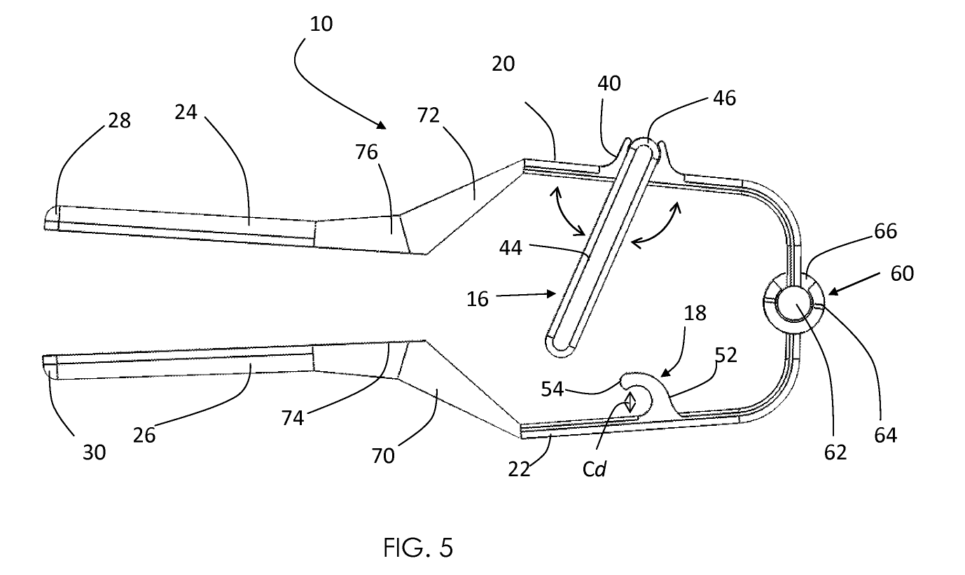

As shown in FIG. 4, the clip members 11 and 13 rotate around the hinge 60 to open and close the jaw members 24 and 26 of the vascular clip 10. The contact surfaces 32 and 34 of each of the jaw members 24 and 26 extend from a proximal point 78 and 79 to a distal end point or tip 28 and 30 respectfully. The contact surfaces 32 and 34 may have a textured grid, or ribbed surface 25 to assist in frictionally adhering a vessel 8 to the surfaces 32 and 34 within the clamp 12. In a first embodiment, the stationary clasp 18 is formed in a hook shape with a rounded exterior surface 52, and an attachment latch 54 that extends a distance Cd from the upper surface 29 of the lower deflection member 22. This distance Cd is substantially the same as the diameter of the barrel cylinder 46. The attachment latch 54 of the clasp 18 extends approximately ⅓ of the distance Cd towards the lower deflection beam 22. In an open and unlocked state the jaws 24 and 26 may be opened or closed with the tension member 16 free to swing about the pivot hinge 40 from the upper deflection member 20. This rotational movement is designated with arrows in FIG. 5. In an open state the deflection beams 20 and 22 are unflexed and extend laterally from the curved frame members 69 and 71 and the vertical supports 21 and 23. In this state even with the jaws 24 and 26 touching one another, the clamping force Fc is essentially zero because the tension member 16 is not latched to the clasp member 18 and therefore the upper and lower deflection members 20 and 22 are not deflected. In this form, the clip 10 is essentially in a static unsecured or unlocked state.

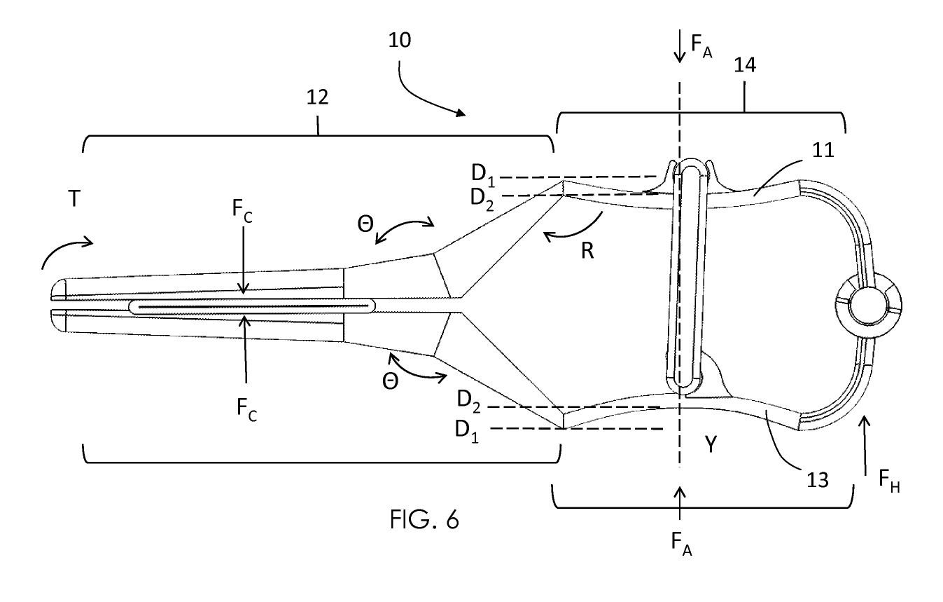

In a closed state, it is an important feature of the invention that when the clip 10 is clamped onto a vessel 8 and is latched all of the clamping force Fc is applied through the vessel 8. A person skilled in the art will realize that a flexural structure such as the clip 10 of the current embodiment may be simulated using computer simulation techniques such as finite element analysis (FEA) in order to predict the deflections and forces in the structure, and to tune the resulting parallelism of the jaws 24 and 26 when the clip 10 is clamped and latched. The jaws 24 and 26 are substantially parallel when the vessel 8 is clamped therein and a gap is shown at the tips 28 and 30 and proximal ends 78 and 79 of the jaws 24 and 26 where the contact surfaces 32 and 34 do not touch. In order to accomplish this, the angle θ is derived through an analysis of the rotation R of the transition support members 70 and 72 with respect to the flexion members 20 and 22 and an analysis of the structural elements and forces within the clamping and frame members 12 and 14. Through the analysis of these forces and the amount of deflection, an adjustment to the angle θ is made to optimize the amount of rotation so that when a vessel 8 is between the contact surfaces 32 and 34, the jaw members 24 and 26 meet and are in parallel to each other and the clamping force Fc is directed to the vessel 8 in the area between the proximal and distal ends of the jaws 24 and 26. As illustrated in FIG. 6, in compressing the deflection members (F_{A}) rotational forces R drive the proximal ends 78 and 79 at each base of the transition members 70 and 72 to approximate causing the tips of the jaws 28 and 30 to separate designated as T until the contact surfaces 32 and 34 are in parallel with one another. Therefore, the contact surfaces 32 and 34 of the jaws 24 and 26 do not contact one another when the tension member 16 is locked and a vessel 8 is sealed and clamped within the jaws 24 and 26. However, the contact surfaces 32 and 34 may be in contact at the tips 28 and 30 when the clamp 12 is closed, the tension member 16 is not latched or in a locked position and there is no vessel 8 within the clamp 12.

In order to seal and clamp a vessel 8 and thereby restrict blood flow the clamping force must be in a range of approximately 50-500 grams of force as described above. The overall dimensions of the frame handle of the vascular clip are approximately 7 mm-25 mm in length and 4 mm-15 mm in height, or the height is approximately one half of the overall length of the clip 10. A jaw leg may be on the order of 1 mm-2 mm in diameter or thickness and the jaw support member 74 and transition member 72 may be 1 mm-3 mm thick to provide support and rigidity to the jaw leg members 24 and 26. The deflection members 20 and 22 may be of a minimal thickness of roughly ½ mm to 2 mm and be flexible thereby when a force is applied perpendicularly to the member shown as F_{A} in FIG. 6, the deflection members 20 and 22 bend or deflect. As shown, the tension member is of a length of approximately 75%-85% of the unflexed length of the clip frame handle 14, and therefore force F_{A} must be applied to the deflection members 20 and 22 to reduce the overall distance between the upper and lower deflection members and provide for the attachment of the tension member 16 to the clasp 18 of the lower deflection member. With an applied force of ˜0 the deflection members 20 and 22 are at rest along datum D₁. By applying the adequate amount of force F_{A} to attach the tension member 16, the deflecting beams 20 and 22 will deflect to a maximum datum of D₂. The length of the tension member 16 determines the clamping force F_{c} realized at the jaws when the tension member 16 is attached and the clip 10 is in a locked position as described in further detail herein.

In the present embodiment, there are two critical criteria for obtaining the desired clamping force Fc as follows;

1. The applied (input) force F_{A} (which is held by the clasp) must be at least greater than the clamping force F_{C}. Therefore the material and geometry must be stiff enough to provide this force F_{C} (at the jaws).

2. The applied (input) force F_{A} must not cause the jaws to spread open.

To be comparable to metallic clips of the prior art and to provide the necessary force to seal and prevent further blood flow through a vessel, the clamping force Fc must be in a range of 50 to 500 grams of force and more specifically in a range of 100 to 300 grams of force. As a starting point, an assumption is made that F_{A}, the applied force for the deflection of the deflecting beams, is located directly between F_{H}, the applied force at the hinge and F_{C} the clamping force, therefore F_{H}=F_{C} and F_{A}=2F_{C}. This is the starting point for the analysis of the flexion. A requirement of the present invention is that the deflection of the deflection beams must be such that the required force (2Fc) causes enough deflection in the beams 20 and 22 to enable the clasp 18 to catch the tension member 16. In order to maintain the clamping force Fc within this acceptable range, an important feature of the present invention is applying force to the deflection beams 20 and 22 to compress the deflection beams 20 and 22 and to lock the tension member 16 and seal a vessel 8 using the clip applier 80 of the present invention.

In a first embodiment, the clasp member 18 is positioned directly opposite the pivot hinge 40 or at a slight offset along axis Y that extends through the upper deflection member pivot hinge 40. The tension member 16 aligns substantially linearly along the Y axis from the pivot hinge 40 and when the deflection members 20 and 22 are compressed the tension member 16 attaches to the clasp member 18 of the lower deflection member 22. The clasp member 18 may have a hook 54, snap fastener or other locking mechanism that facilitates the attachment of the barrel cylinder 46 or other attachment mechanism of the tension member 16 to releasably secure the tension member 16 to the lower deflection member 22 and hold the deflection members 20 and 22 in compression.

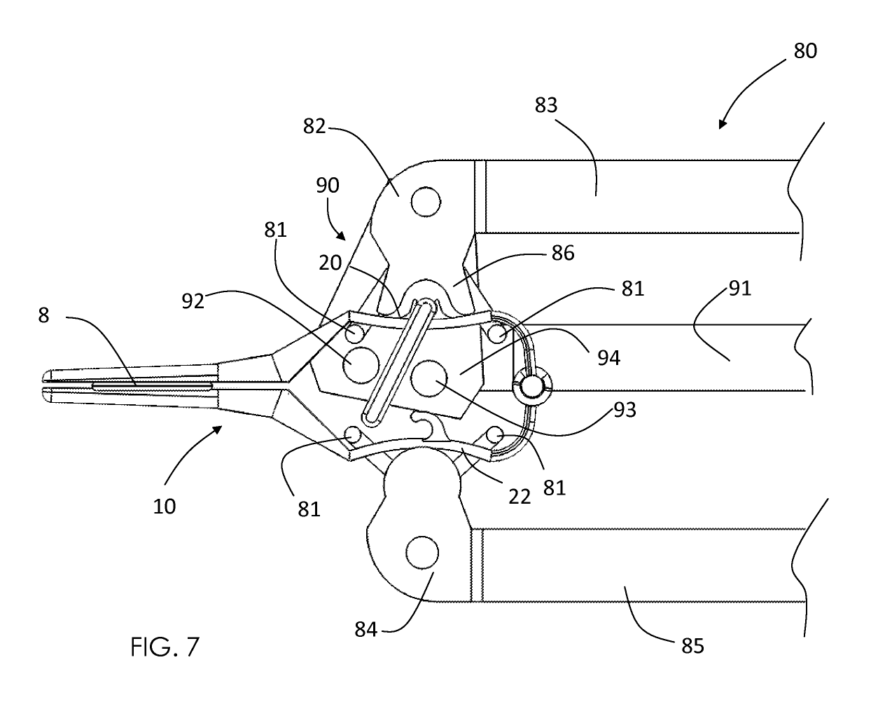

As shown in FIG. 7, clip applier forceps 80 securely hold the vascular clip 10 to open, maneuver and manipulate the clip 10 to surround and clamp a vessel 8. A set of engagement pins 81 mounted to each end effector 82 and 84 engage the inside of each of the deflection members and provide for separating the clip members 11 and 13 to open and close the clip 10. A pair of forceps arms 83 and 85 provides for releasing of the clip 10 when the clip is properly positioned to clamp a vessel 8. Compression force to flex the deflection members 20 and 22 and apply force to the clamp 12 is performed using end effectors 82 and 84 that include a contoured surface that is complimentary to the outer surface of deflection member. As shown, the upper end effector 82 may include a cup shaped member 86 that interfaces with the pivot hinge 40 to assist in holding and maneuvering the clip 10. The lower end effector 84 may include a rounded bulged surface 88 to apply force to the center of the deflection member 22 that, along with the compression of the upper end effector 84, reduces the distance between the deflection members 20 and 22 to a distance that is less than the length of the tension member 16, so that the tension member 16 will reach and latch to the clasp 18. Other end effector features may serve as locaters and connectors to securely hold the clip 10 until it is finally positioned and clamped to seal a vessel 8.

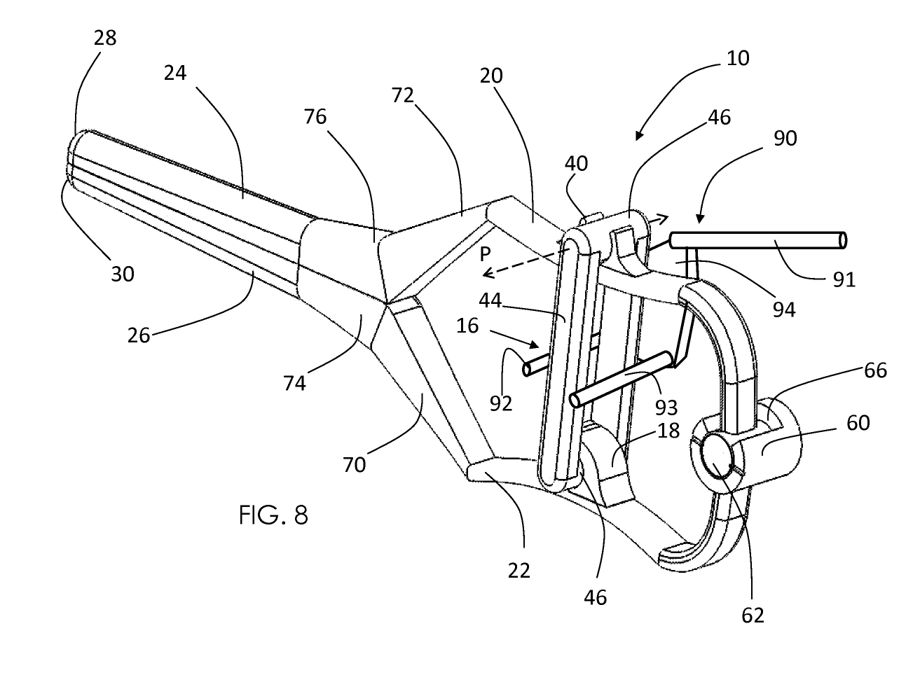

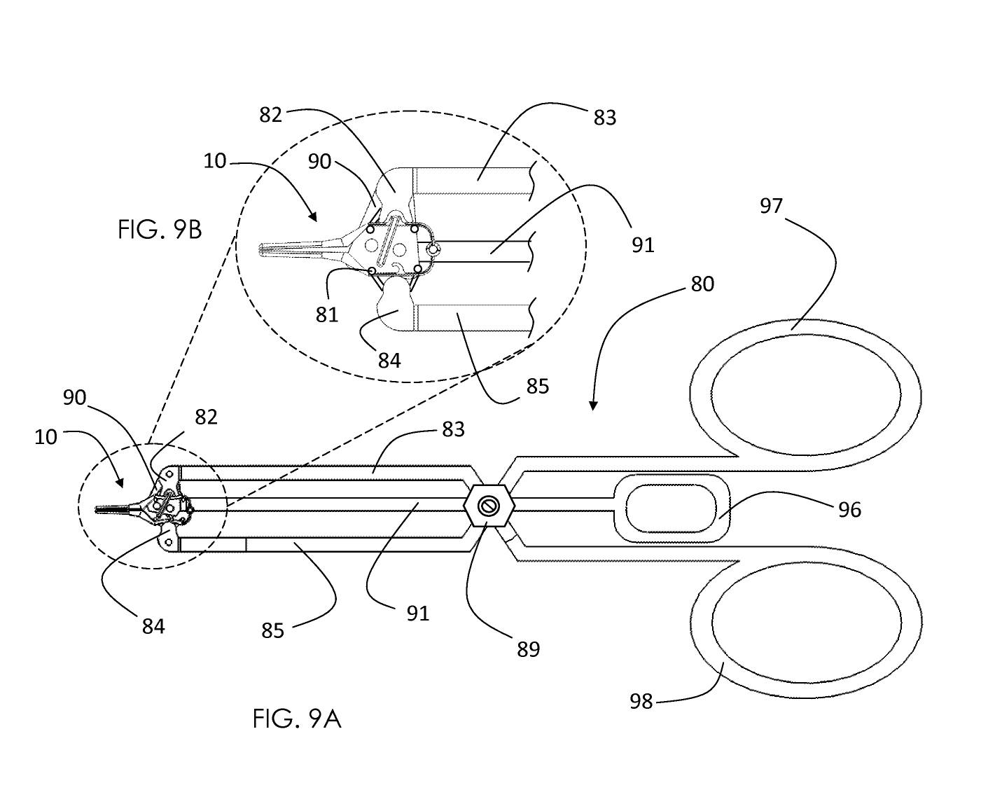

Importantly, the tension member 16 in rotating about the pivot axis P provides for a latch actuator 90 of the clip applier 80 of the present invention to manipulate the tension member 16 to a latched and unlatched position by pushing the member 16 into place to lock the clip 10, or alternatively pulling the member 16 out of the clasp 18 to unlock the clip 10. The actuator arm 91 extends to a control handle 96. The latch actuator 90 is shown in isolation in FIG. 8 showing the actuator arms 92 and 93 extending out from a base plate 94 of the latch actuator 90 to align on either side of the tension member 16 to push or pull the tension member 16. By moving the actuator trigger 96, shown in FIG. 9A, the tension member 16 may be moved from a locked to an unlocked position using the actuator arms 92 and 93 by swinging the tension arm 16 around the pivot axis P while the end effectors 82 and 84 may maintain pressure on the clip 10 and thereby hold a vessel 8 within the clamp 12 while locking and unlocking the clip 10. This feature is critical to allow a surgeon to place the clip 10 and determine if there is a cessation of bleeding. If there is still blood flow or if the clip is otherwise positioned in an undesirable location in the anatomy, the surgeon may unlock the clip 10 using the actuator trigger 96 and reposition the clip 10 on the vessel 8 at the desired anatomical location. The upper and lower compression handles 97 and 98 may be manipulated to hold, maneuver, compress, and release the clip 10. As shown in FIG. 9B, the clip 10 may be in a non-flexed position and be held and manipulated by the clip applier 80.

As shown in FIGS. 9A and 9B, the very small size of the vascular clip 10 requires that the clip applier 80 must hold, compress and adjust the clip 10 from an open to a closed position. The clip applier 80 of the present invention is further of an ergonomic design that provides for minimal force and acuity to be needed to efficiently maneuver and attach a clip 10. The compression using the clip applier flexes the deflection members 20 and 22 and these members 20 and 22 remain flexed when the tension member 16 is secured by the clasp 18 to the vessel 8. The amount of deflection is primarily determined by the material thickness and modulus of elasticity of the plastic of the deflection members 20 and 22 and the length of the tension member 16.

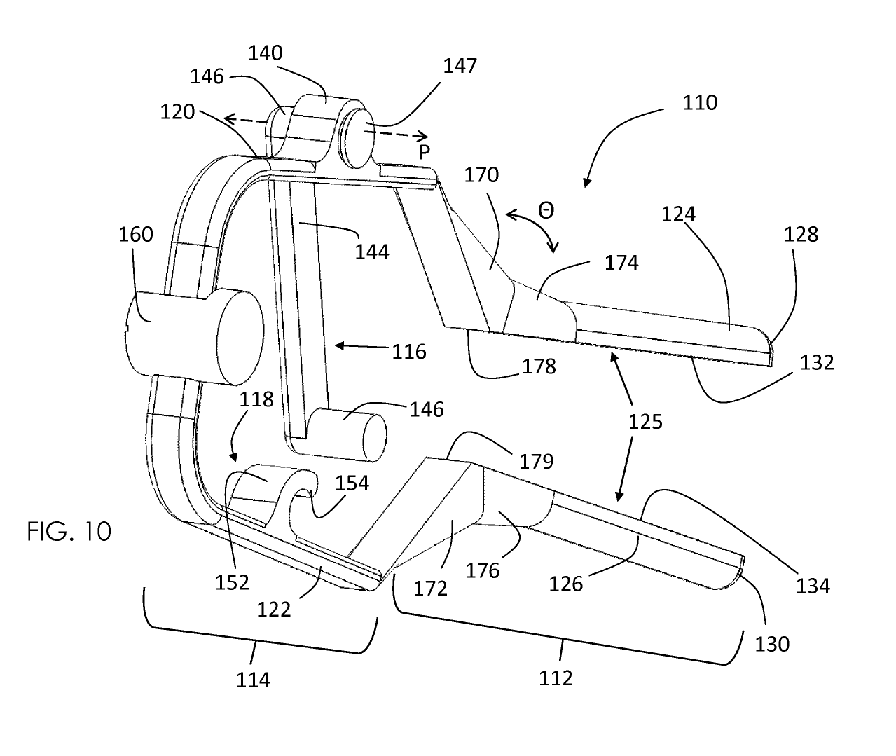

In further embodiments such as shown in FIG. 10, the vascular clip 110 is formed with a single sided tension member 116 as shown. In this embodiment, the tension member 116 is secured within an enclosed full-round pivot fastener 140. The retaining cylinder 146 may be secured with an oversized mushroom flange 147 or boss, or using a heat stake or heat treatment to deform the plastic and retain the tension arm 116 within the enclosed pivot fastener 140. The tension arm 144 may be thickened to provide additional support when securing the arm 144 to the clasp 118 and to support the grip of the applier 80 in manipulating the tension member 116 to open and close the clamp 112. The jaw members 124 and 126 may be formed similarly to previous embodiments with thickened transition support members 170 and 172 and stiffening members 174 and 176, the jaw members 124 and 126 extending from proximal points 178 and 179 where the frame member 114 meets the clamp member 112 to the jaw tips 128 and 130. The contact surfaces 132 and 134 of the jaws 24 and 26 may also be similarly formed with a grid or grooved surface 125. The pivot fastener 140 provides for the tension arm 116 to rotate around pivot axis P and be fastened to the clasp member 118. The clasp 118 is similarly formed with a rounded arched surface 152 and a nubbed or protruding end attachment latch 154. The tension arm 144 is also similarly formed with a barrel cylinder 146 that may be secured within nub or protrusion of the attachment latch 154. The angle θ is determined through a similar analysis to the prior embodiments to minimize the amount of rotation so that when a vessel 8 is between the contact surfaces 132 and 134, the jaw members 124 and 126 meet and are in parallel to each other and the clamping force Fc is directed to the vessel 8 in the area between the proximal and distal ends of the jaws 124 and 126 as previously described.

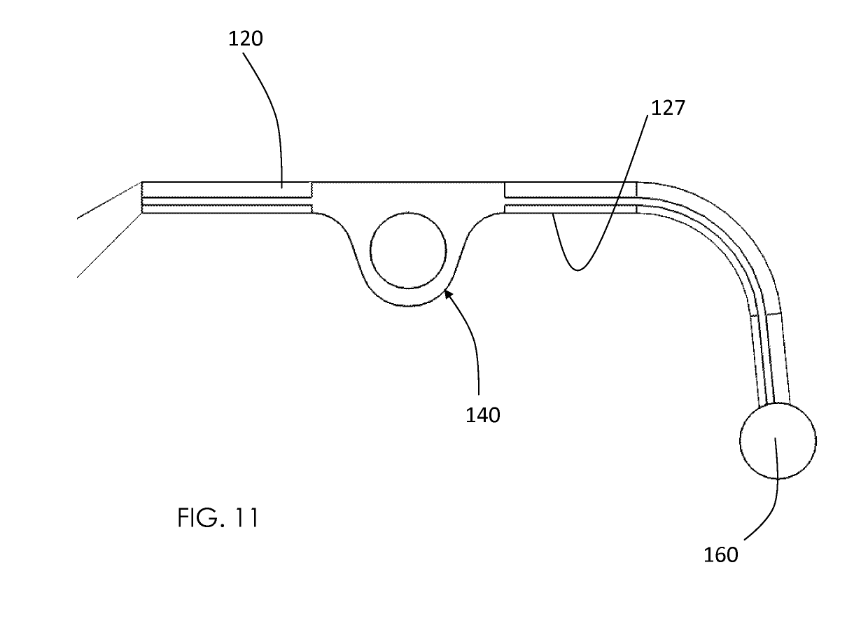

In further embodiments, the pivot fastener 140 may extend from the interior surface 129 of a deflection member 120 as shown in FIG. 11. A shorter tension member may then be used to provide adequate clamping force Fc in securing the tension arm to the clasp. Other fasteners or retainers that provide for the tension arm to pivot or swing are contemplated within the scope of the present invention.

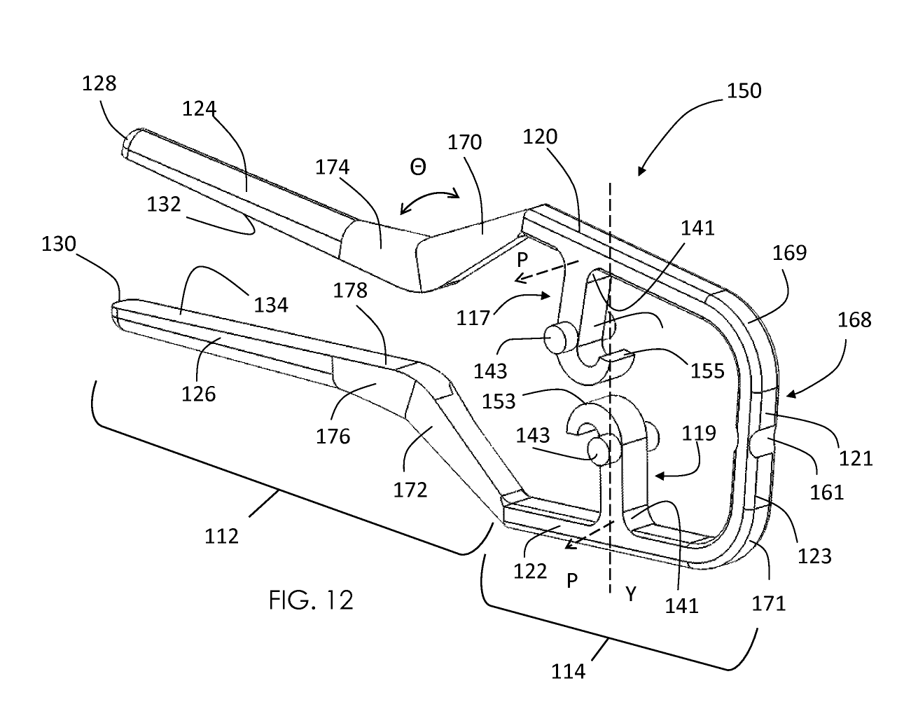

In further embodiments of the vascular clip 150 such as shown in FIG. 12, the locking mechanism may be formed from a pair of opposing clasps or hooks 117 and 119 that extend from the interior surfaces 127 and 129 of each of the deflection members 120 and 122. The hook fasteners 117 and 119 may be the same size and dimension or either fastener may be longer than the other fastener with each fastener affixed to each deflection member 120 and 122 using a flexible hinge 141 that provides for rotational movement of the hook fasteners 117 and 119 around the pivot axis P. To lock and secure the vascular clip 150, the upper and lower hook fasteners 117 and 119 overlap so that by using a clip applier 80 to compress each of the deflection members 120 and 122 the outer surfaces 153 of each of the fasteners 117 and 119 flex about the flexible hinges 141, make contact with one another and brush along each of the surfaces 153 until the latching nubs or protrusions 155 engage and interlock and the fasteners 117 and 119 flex back approximately into their initial positions along the Y axis locking the jaw members 124 and 126 with sufficient force Fc. The hook clasps or fasteners 117 and 119 may further provide a clip applier access point 143 to provide for the attachment of surgical forceps 80 or another applier tool to open, position and close the vascular clip 150 and reopen as necessary and reposition in the proper anatomical location to seal a vessel 8 and stop blood flow. The angle θ is also determined through a similar analysis to the prior embodiments to minimize the amount of rotation so that when a vessel 8 is clamped between the contact surfaces 132 and 134, the jaw members 124 and 126 meet and are parallel to each other and the clamping force Fc is directed to the vessel 8 in the area between the proximal and distal ends of the jaws 124 and 126 as previously described. Alternative applier mechanisms may use tensile fiber, such as dyneema with round or bulged ends to encircle the clip frame and clamp and secure the vascular clip in a locked position. Access points may be positioned on the tension member or clasp to provide for a clip applier to grasp the tension member and swing or otherwise adjust the member around a pivot axis to position the clip in a closed and secure location, and re-open and re-position and re-close and secure the clip to properly seal the vessel and stop blood flow.

In this embodiment the vascular clip 150 is formed as a single unitary piece with a living hinge 161 that provides for the clip 150 to open and close. The living hinge 161 may be formed along the vertical support 168 of the frame 114 with the hinge region 161 formed by diminishing the amount of material within this region to provide for bending of the vertical support 168 in opening and closing the jaw members 124 and 126. The upper and lower members 121 and 123 of the vertical support 168 may also be of an increased thickness to rigidly support the deflection beams 120 and 122 in compression. The vertical members 121 and 123 extend to curved members 169 and 171 that are formed substantially perpendicular to the vertical support structure 168 at an angle approaching 90° and in further embodiments may be of any angle in a range of approximately 45° to 130°.

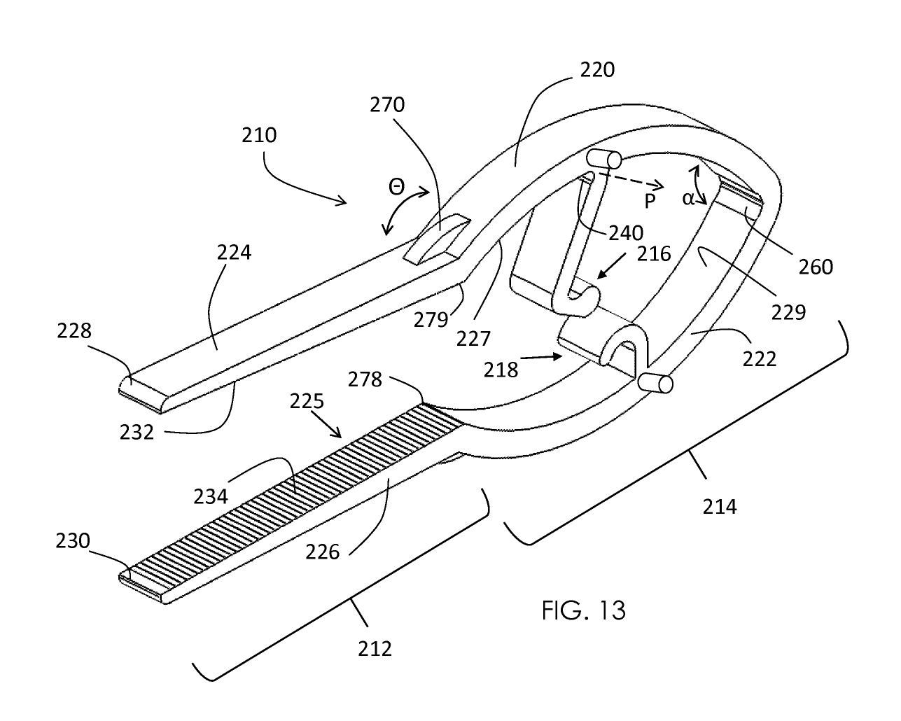

In further embodiments, the vascular clip 210 is formed with a frame member 214 in a crescent, elliptical or semicircular shape as shown in FIG. 13. In this embodiment the vascular clip 210 may have a lower vertical profile and a smooth overall shape that is atraumatic when inside the body. The jaw members 224 and 226 are similar to prior embodiments, and extend from proximal transition points 278 and 279 between the frame 214 and the clamp 212 to the jaw tips 228 and 230 with similar contact surfaces 232 and 234 that may be gridded or ribbed 225. However, instead of transition support and stiffening members, a support rib 270 may extend from the transition points 278 and 279 between the clamp 212 and frame 214 to stiffen and hold the jaw members 224 and 226 substantially rigid during compression of the frame members 220 and 222.

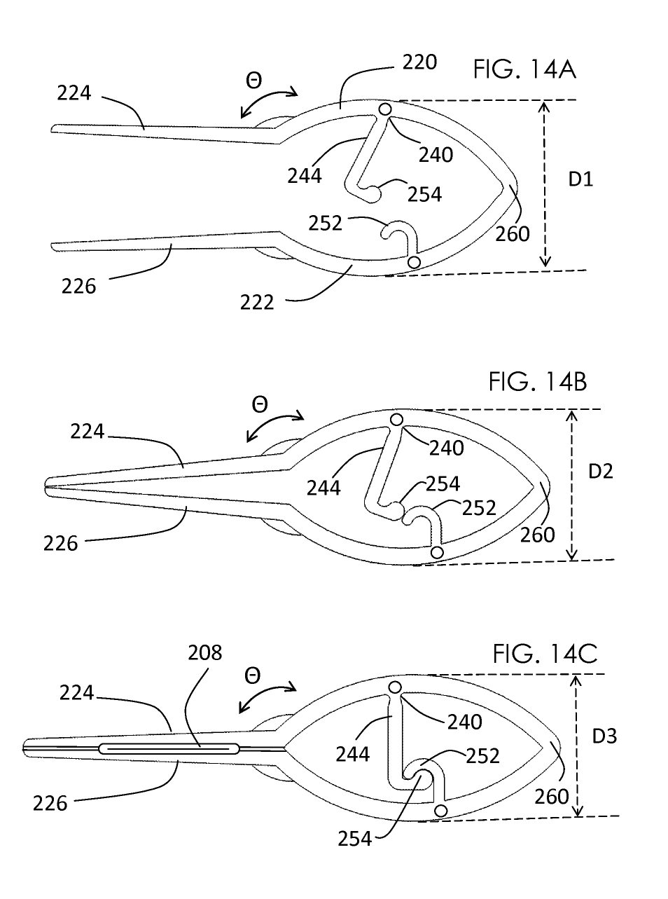

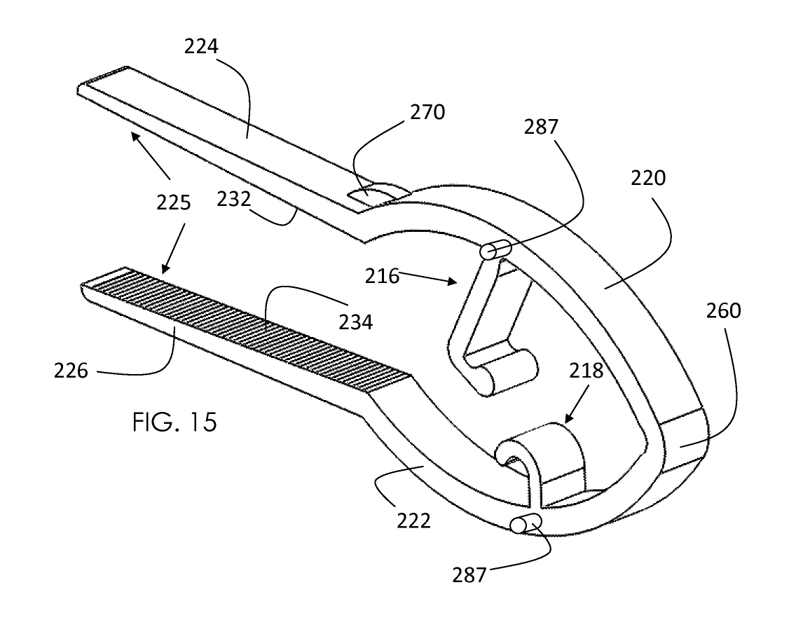

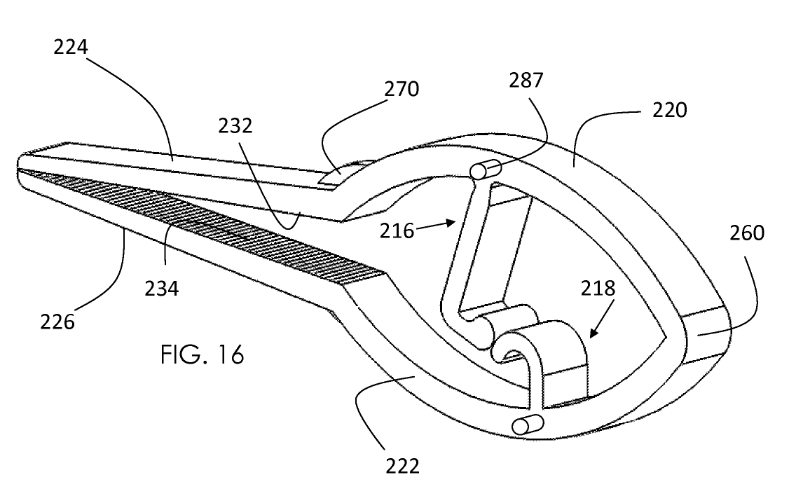

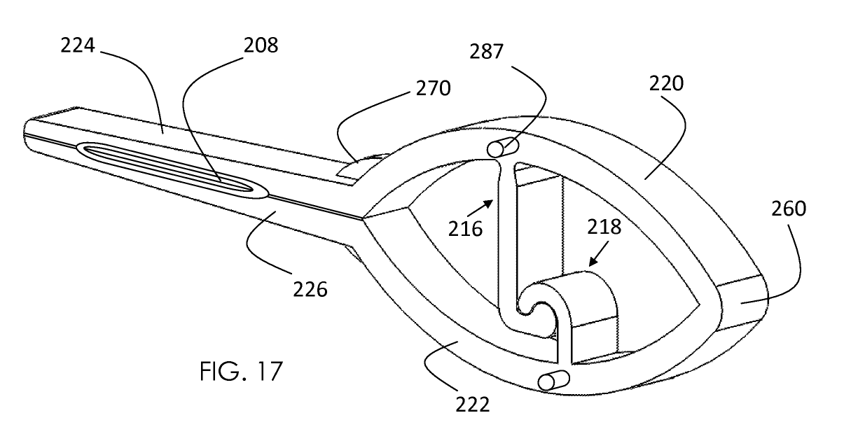

The vascular clip 210 may be formed as a single unitary piece with a living hinge 260 connecting the upper and lower deflection members 220 and 222 and providing for the clip 210 to be opened and closed. The living hinge 260 may allow the clip 210 to open at an angle α that may be in a range of approximately 30° to 160° and more specifically to a range of 45° to 80° to provide for the applier tool 80 to grip and maneuver the clip members around a vessel 208 for clamping. The tension member 216 may be affixed to the interior surface 227 of the upper deflection member 220 and extend using a flexible hinge 240 to swing around pivot axis P. It will be appreciated by one skilled in the art that this embodiment may alternatively be constructed with a hinged pivot with a tension member and latch as shown in previous embodiments. A clip applier 80 may grip the upper and lower deflection members 220 and 222 to hold and maneuver the clip 210 to the proper position and then compress the clip 210 and shorten the distance D between the deflection members 220 and 222 to have the tension member 216 reach and connect to the clasp 218 as shown in FIGS. 14A-14C. As the clip 210 is compressed the distance D is shortened from a distance D1 to an intermediate distance D2 and finally to a latched distance D3. The derived angle θ for this embodiment may provide for the tips of the clamp 212 to toe-in and meet when the clamp 212 is closed before the vessel 208 is clamped within the clip 210 and for aligning the jaw members 224 and 226 in parallel such that the contact surfaces 232 and 234 not touching when a vessel 208 is within the clamp 212. In FIG. 15, the clip 210 is shown in an open position with access points 287 on one or both of the upper and lower deflection members 220 and 222 to attach a clip applier to hold the clip 210 and control the opening and closing of the clip 210. As shown is FIG. 16, in a partially closed position, the derived angle θ provides for the tips 228 and 230 to mate first when no vessel 208 is within clamp 212, and have the jaw members 224 and 226 align in parallel when a vessel 208 is clamped within the jaw members 224 and 226 as shown in FIG. 17.

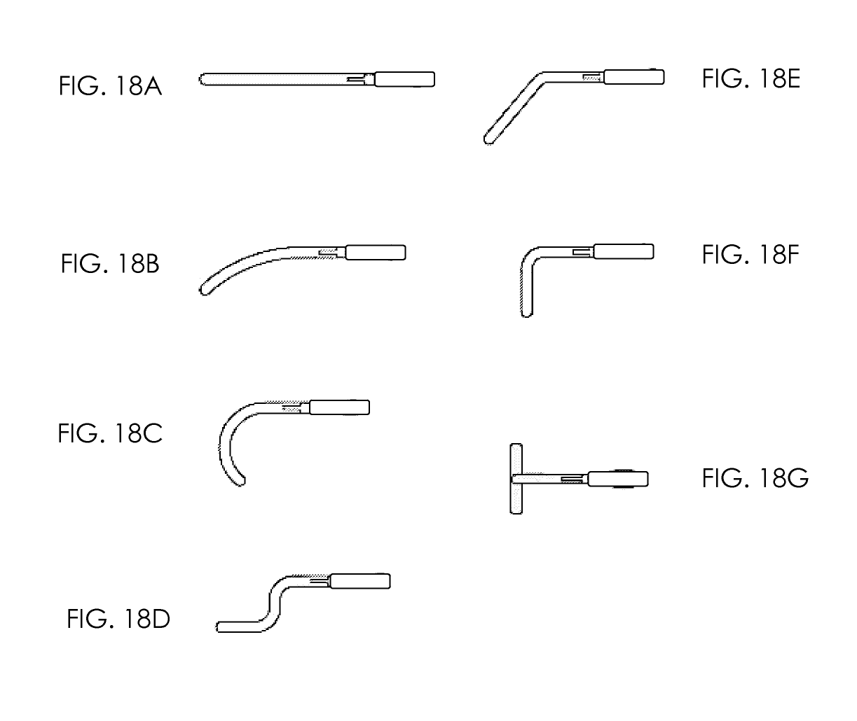

In any of the embodiments described herein the vascular clip may be formed with jaws that are curved, rounded, angled or have perpendicular or other angular extensions from the clamp surface 12 as shown in FIGS. 18A-18G. The optional jaw geometries provide for a selection of the proper tip style to accommodate surgical and anatomical requirements. For example, the aneurysm may be within a portion of the brain that is particularly difficult to access, and the proper jaw clamping surface provides for the insertion around critical anatomical areas and assists in supporting the vessel to properly lock and secure the clip to stop blood flow. The properly secured clip of the PEEK or other biocompatible material provides for zero artifacts and interference in MRI, CT and other diagnostic equipment. This significant benefit allows for improved analysis and reduces post-operative complications.

The invention has been described in detail with particular reference to certain preferred embodiments thereof, but it will be understood that variations and modifications can be effected within the spirit and scope of the invention.

What is claimed is:

1. A re-attachable vascular clip comprising:
two jaws having clamping surfaces;
two flexible members manipulating the two jaws to close;
a tension member latching the two flexible members to lock the two jaws in a closed position; and
wherein unlatching the tension member unlocks the two jaws.

2. The re-attachable vascular clip according to claim 1 wherein the tension member is pivotably attached to at least one of the two flexible members.

3. The re-attachable vascular clip according to claim 1 wherein the tension member further comprising a first and second tension arm.

4. The re-attachable vascular clip according to claim 1 wherein the tension member further comprising a single tension arm.

5. The re-attachable vascular clip according to claim 1 wherein the tension member further comprising opposing clasps.

6. The re-attachable vascular clip according to claim 1 wherein the tension member further comprising a clip actuator manipulating the tension member to latch and unlatch the two flexible members.

7. The re-attachable vascular clip according to claim 1 wherein the two jaws close at a clamping force in a range of 50 to 500 grams of force.

8. The re-attachable vascular clip according to claim 1 wherein the vascular clip is a biocompatible plastic material.

9. The re-attachable vascular clip according to claim 8 wherein the vascular clip produces no imaging interference.

10. An aneurysm clip producing minimal interference in imaging comprising:
a compressible frame of a plastic material;
a clamping member extending from the compressible frame; and
wherein compressing the frame produces forces at the clamping member in a range of 50 to 500 grams of force.

11. The aneurysm clip producing minimal interference in imaging of claim 10 further comprising a latching member holding the frame in a compressed state.

12. The aneurysm clip producing minimal interference in imaging of claim 11 wherein the latching member releases the frame from a compressed state.

13. The aneurysm clip producing minimal interference in imaging of claim 11 wherein the latching member pivots from the frame.

14. The aneurysm clip producing minimal interference in imaging of claim 11 wherein the latching member comprises first and second clasps.

15. The aneurysm clip producing minimal interference in imaging of claim 10 wherein the frame is of a substantially rectangular shape.

16. The aneurysm clip producing minimal interference in imaging of claim 10 wherein the frame is of a substantially elliptical shape.

17. The aneurysm clip producing minimal interference in imaging of claim 10 wherein the clamping member is one of at least a curved, rounded, and angled shape.

18. The aneurysm clip producing minimal interference in imaging of claim 10 wherein the clamping member further comprises an angular extension.

19. A method of applying a zero artifact vascular clip to a vessel to cease blood flow, comprising the steps of:
locating the open jaws of a vascular clip around a vessel;
closing the jaws of the vascular clip around the vessel;
compressing a frame affixed to the jaws to apply adequate force to the vessel to cease blood flow;
locking a tension member using an actuator of a clip applier to hold the frame in compression.

20. The method of applying a zero artifact vascular clip to a vessel to cease blood flow of claim 19 further comprising the steps of:
unlocking the tension member;
decompressing the frame affixed to the jaws;
opening the jaws of the vascular clip;
repositioning the vascular clip around the vessel and closing the jaws of the vascular clip;
compressing the frame affixed to the jaws to apply adequate force to the vessel to cease blood flow;
locking the tension member using the actuator of the clip applier to hold the frame in compression.
